(19) United States
(12) Patent Application Publication
(10) Pub. No.: US 2012/0094955 A1
(43) Pub. Date: Apr. 19, 2012

(54) METHOD OF DISSOLVING AN OXIDIZED POLYSACCHARIDE IN AN AQUEOUS SOLUTION

(75) Inventor: Mark E. Wagman, Wilmington, DE (US)

(73) Assignee: ACTAMAX SURGICAL MATERIALS LLC, Berkeley, CA (US)

(21) Appl. No.: 13/263,379

(22) PCT Filed: Apr. 9, 2010
(86) PCT No.: PCT/US10/30476

§ 371 Date: Oct. 7, 2011

Related U.S. Application Data
(60) Provisional application No. 61/167,877, filed on Apr. 9, 2009.
(60) Provisional application No. 61/167,879, filed on Apr. 9, 2009.
(60) Provisional application No. 61/187,881, filed on Jun. 17, 2009.

(51) Int. Cl. A61K 31/721; A61K 47/26; A61P 43/00; C09J 105/02

(52) U.S. Cl. 514/59; 106/217.6; 514/777

(57) ABSTRACT

A method of dissolving an oxidized polysaccharide in an aqueous solution using an oligomer additive is described. The resulting aqueous solution of the oxidized polysaccharide may be used in combination with an aqueous solution comprising an amine-containing component to prepare hydrogel tissue adhesives and sealants for medical and veterinary applications, such as wound closure, supplementing or replacing sutures or staples in internal surgical procedures such as intestinal anastomosis and vascular anastomosis, tissue repair, ophthalmic procedures, drug delivery, and to prevent post-surgical adhesions.

### CROSS-REFERENCE TO RELATED APPLICATIONS

This application claims priority under 35 U.S.C. §119 from U.S. Provisional Application Ser. Nos. 61/167,877, 61/167,881, and 61/167,879, all of which were filed on Apr. 9, 2009.

### FIELD OF THE INVENTION

The invention relates to the field of medical adhesives. More specifically, the invention relates to a method of dissolving an oxidized polysaccharide in an aqueous solution using an oligomer additive to enhance the dissolution.

### BACKGROUND OF THE INVENTION

Tissue adhesives have many potential medical applications, including wound closure, supplementing or replacing sutures or staples in internal surgical procedures, adhesion of synthetic onlays or inlays to the cornea, drug delivery devices, and as anti-adhesion barriers to prevent post-surgical adhesions. Conventional tissue adhesives are generally not suitable for a wide range of adhesive applications. For example, cyanoacrylate-based adhesives have been used for topical wound closure, but the release of toxic degradation products limits their use for internal applications. Fibrin-based adhesives are slow curing, have poor mechanical strength, and pose a risk of viral infection. Additionally, fibrin-based adhesives do not bond covalently to the underlying tissue.

Several types of hydrogel tissue adhesives have been developed, which have improved adhesive and cohesive properties and are nontoxic. These hydrogels are generally formed by reacting a component having nucleophilic groups with a component having electrophilic groups, which are capable of reacting with the nucleophilic groups of the first component, to form a crosslinked network via covalent bonding. A number of these hydrogel tissue adhesives are prepared using an oxidized polysaccharide containing aldehyde groups as one of the reactive components (see for example, Kodokian et al., copending and commonly owned U.S. Patent Application Publication No. 2006/0078536, Goldmann, U.S. Patent Application Publication No. 2005/0002893, and Nakajima et al., U.S. Patent Application Publication No. 2008/0319101). However, the instability of oxidized polysaccharides in aqueous solution limits their shelf-life for commercial use. Moreover, oxidized polysaccharides dissolve very slowly when added to an aqueous solution (i.e., many hours at elevated temperature to dissolve), making the preparation of the aqueous solution from the more stable solid form at the time of use impractical.

Therefore, the need exists for a method to enhance the dissolution of oxidized polysaccharides in aqueous solution to enable the preparation of the solution from the solid form at the time of use.

### SUMMARY OF THE INVENTION

The present invention addresses the above need by providing a method of dissolving an oxidized polysaccharide in aqueous solution. The method utilizes an oligomer additive, which enhances the dissolution of the oxidized polysaccharide.

Accordingly, in one embodiment the invention provides a method of dissolving an oxidized polysaccharide in an aqueous solution comprising the steps of:
a) providing at least one oxidized polysaccharide in dry powder form, said oxidized polysaccharide containing aldehyde groups, and having a weight-average molecular weight of about 1,000 to about 1,000,000 Daltons, and an equivalent weight per aldehyde group of about 65 to about 1500 Daltons;
b) providing an aqueous solution;
c) providing at least one oligomer of the formula:

R1-(PS)—R2
wherein:
   (i) PS is a linear polymeric segment comprising ethylene oxide monomers or a combination of ethylene oxide and propylene oxide monomers, wherein said ethylene oxide monomers comprise at least about 50 weight percent of said polymeric segment;
   (ii) R1 is at least one nucleophilic group capable of reacting with aldehyde groups to form at least one reversible covalent bond;
   (iii) R2 is at least one functional group which is not capable of reacting with an aldehyde, a primary amine, a secondary amine, or R1 to form a covalent bond, such that said oligomer does not induce gelation when mixed in the aqueous solution with (a);
   (iv) said oligomer has a weight-average molecular weight of about 200 to about 4,000 Daltons; and
   (v) said oligomer is water soluble;
d) combining (a), (b), and (c) in any order to form a heterogeneous mixture; and
e) agitating the mixture obtained in step (d) to effect dissolution of the oxidized polysaccharide to obtain an aqueous solution of the oxidized polysaccharide.

In another embodiment the invention provides an aqueous composition comprising:
a) water;
b) at least one oxidized polysaccharide containing aldehyde groups, said oxidized polysaccharide having a weight-average molecular weight of about 1,000 to about 1,000,000 Daltons and having an equivalent weight per aldehyde group of about 65 to about 1500 Daltons; and
c) at least one oligomer of the formula:

R1-(PS)—R2
wherein:
   (i) PS is a linear polymeric segment comprising ethylene oxide monomers or a combination of ethylene oxide and propylene oxide monomers, wherein said ethylene oxide monomers comprise at least about 50 weight percent of said polymeric segment;
   (ii) R1 is at least one nucleophilic group capable of reacting with aldehyde groups to form at least one reversible covalent bond;
   (iii) R2 is at least one functional group which is not capable of reacting with an aldehyde, a primary amine, a secondary amine, or R1 to form a covalent bond, such that said oligomer does not induce gelation when mixed in the aqueous solution with (b);
   (iv) said oligomer has a weight-average molecular weight of about 200 to about 4,000 Daltons; and
   (v) said oligomer is water soluble.

### DETAILED DESCRIPTION

As used above and throughout the description of the invention, the following terms, unless otherwise indicated, shall be defined as follows:

The term “dissolution” refers to the process of dissolving a solid substance in a solvent to yield a solution.

The term “oxidized polysaccharide” refers to a polysaccharide which has been reacted with an oxidizing agent to introduce aldehyde groups into the molecule.

The term “equivalent weight per aldehyde group” refers to the molecular weight of the oxidized polysaccharide divided by the number of aldehyde groups introduced in the molecule.

The term “water-dispersible, multi-arm polyether amine” refers to a polyether having three or more polymer chains (“arms”), which may be linear or branched, emanating from a central structure, which may be a single atom, a core molecule, or a polymer backbone, wherein at least three of the branches (“arms”) are terminated by at least one primary amine group. The water-dispersible, multi-arm polyether amine is water soluble or is able to be dispersed in water to form a colloidal suspension capable of reacting with a second reactant in aqueous solution or dispersion.

The term “dispersion” as used herein, refers to a colloidal suspension capable of reacting with a second reactant in an aqueous medium.

The term “polyether” refers to a polymer having the repeat unit [—O—R]—, wherein R is a hydrocarbyl group having 2 to 5 carbon atoms. The polyether may also be a random or block copolymer comprising different repeat units having different R groups.

The term “hydrocarbylene group” refers to a divalent group formed by removing two hydrogen atoms, one from each of two different carbon atoms, from a hydrocarbon.

The term “crosslink” refers to a bond or chain of atoms attached between and linking two different polymer chains. The term “crosslink density” is herein defined as the reciprocal of the average number of chain atoms between crosslink connection sites.

The term “% by weight”, also referred to herein as “wt %” refers to the weight percent relative to the total weight of the solution or dispersion, unless otherwise specified.

The term “nucleophilic group” as used herein refers to an atom or a group of atoms within a molecule that form a chemical bond by donating electrons, i.e., a nucleophilic group is an electron donating group.

The term “functional group” as used herein refers to an atom or a group of atoms within a molecule that undergo characteristic chemical reactions.

The term “reversible covalent bond” as used herein refers to a covalent bond that undergoes a reversible reaction.

The term “reversible reaction” as used herein refers to a chemical reaction that can be made to proceed in either direction (i.e., forward or reverse) by changing physical conditions.

The term “covalent bond” as used herein refers to a type of chemical bonding that is characterized by the sharing of pairs of electrons between atoms.

The term “water soluble” as used herein means that a material is capable of being dissolved in water at a concentration of at least 1 weight percent and remains in solution at a temperature of 18 to 25° C. and atmospheric pressure (i.e., 740 to 760 mm of mercury).

The term “hydrogel” refers to a water-swellable polymeric matrix, consisting of a three-dimensional network of macromolecules held together by covalent crosslinks, that can absorb a substantial amount of water to form an elastic gel.

The term “PEG” as used herein refers to polyethylene glycol.

The term “M_{w}” as used herein refers to the weight-average molecular weight.

The term “Mₙ” as used herein refers to the number-average molecular weight.

The term “medical application” refers to medical applications as related to humans and animals.

The meaning of abbreviations used is as follows: “min” means minute(s), “h” means hour(s), “sec” means second(s), “d” means day(s), “mL” means milliliter(s), “L” means liter(s), “μL” means microliter(s), “cm” means centimeter(s), “mm” means millimeter(s), “μm” means micrometer(s), “mol” means mole(s), “mmol” means millimole(s), “g” means gram(s), “mg” means milligram(s), “wt %” means percent by weight, “mol %” means mole percent, “Vol” means volume, “v/v” means volume per volume, “Da” means Dalton(s), “kDa” means kiloDalton(s), the designation “10K” means that a polymer molecule possesses a number-average molecular weight of 10 kiloDaltons, “M” means molarity, “Pa” means pascal(s), “kPa” means kilopascal(s), “mTorr” means milliTorr”, “¹H NMR” means proton nuclear magnetic resonance spectroscopy, “ppm” means parts per million, “PBS” means phosphate-buffered saline, “RT” means room temperature, “rpm” means revolutions per minute, “psi” means pounds per square inch.

A reference to “Aldrich” or a reference to “Sigma” means the said chemical or ingredient was obtained from Sigma-Aldrich, St. Louis, Mo.

Disclosed herein is a method of dissolving an oxidized polysaccharide in an aqueous solution. In the method, an oligomer is added to the aqueous solution to enhance the dissolution of the oxidized polysaccharide. The aqueous solution of the oxidized polysaccharide may be used in combination with an aqueous solution comprising an amine-containing component to prepare hydrogel tissue adhesives and sealants for medical and veterinary applications, including, but not limited to, wound closure, supplementing or replacing sutures or staples in internal surgical procedures such as intestinal anastomosis and vascular anastomosis, tissue repair, ophthalmic procedures, drug delivery, and to prevent post-surgical adhesions.

### Oxidized Polysaccharides

Oxidized polysaccharides useful in the invention include, but are not limited to, oxidized derivatives of: dextran, carboxymethyldextran, starch, agar, cellulose, hydroxyethylcellulose, carboxymethylcellulose, pullulan, inulin, levan, and hyaluronic acid. The starting polysaccharides are available commercially from sources such as Sigma Chemical Co. (St. Louis, Mo.). Typically, polysaccharides are a heterogeneous mixture having a distribution of different molecular weights, and are characterized by an average molecular weight, for example, the weight-average molecular weight (M_{w}), or the number average molecular weight (Mₙ), as is known in the art. Suitable oxidized polysaccharides have a weight-average molecular weight of about 1,000 to about 1,000,000 Daltons, more particularly about 3,000 to about 250,000 Daltons, more particularly about 5,000 to about 100,000 Daltons, and more particularly about 10,000 to about 60,000 Daltons. In one embodiment, the oxidized polysaccharide is oxidized dextran, also referred to herein as dextran aldehyde.

Oxidized polysaccharides may be prepared by oxidizing a polysaccharide to introduce aldehyde groups using any suitable oxidizing agent, including but not limited to, periodates, hypochlorites, ozone, peroxides, hydroperoxides, persulfates, and percarbonates. For example, the polysaccharide may be oxidized by reaction with sodium periodate as described by Mo et al. (J. Biomater. Sci. Polymer Edn. 11:341-351, 2000). The polysaccharide may be reacted with different amounts of periodate to give polysaccharides with different degrees of oxidation and therefore, different amounts of aldehyde groups. Additionally, the oxidized polysaccharide may be prepared using the method described by Cohen et al. (copending and commonly owned International Patent Application Publication No. WO 2008/133847). That method of making an oxidized polysaccharide comprises a combination of precipitation and separation steps to purify the oxidized polysaccharide formed by oxidation of the polysaccharide with periodate, as described in detail in the Examples herein below, and provides an oxidized polysaccharide with very low levels of iodine-containing species. The degree of oxidation, also referred to herein as the oxidation conversion, of the oxidized polysaccharide may be determined using methods known in the art. For example, the degree of oxidation of the oxidized polysaccharide may be determined using the method described by Hofreiter et al. (Anal Chem. 27:1930-1931, 1955). In that method, the amount of alkali consumed per mole of dialdehyde in the oxidized polysaccharide, under specific reaction conditions, is determined by a pH titration. Alternatively, the degree of oxidation of the oxidized polysaccharide may be determined using nuclear magnetic resonance (NMR) spectroscopy, as described in detail in the Examples herein below. In one embodiment, the equivalent weight per aldehyde group of the oxidized polysaccharide is from about 65 to about 1500 Daltons, more particularly from about 90 to about 1500 Daltons.

### Oligomer Additives

The oligomer additive serves to enhance the dissolution of the oxidized polysaccharide in an aqueous solution. Suitable oligomer additives have the general formula:

R1-(PS)—R2 (1)

wherein: PS is a linear polymeric segment comprising ethylene oxide monomers or a combination of ethylene oxide and propylene oxide monomers, wherein the ethylene oxide monomers comprise at least about 50 wt % of the polymeric segment, more particularly at least about 60 wt %, more particularly at least about 70 wt %, more particularly at least about 80 wt %, more particularly at least about 90 wt %, and more particularly 100 wt % of the polymeric segment. PS may comprise random or block copolymers of ethylene oxide and propylene oxide. The polymeric segment may also comprise a linker to attach R1 and R2 to the ends of the polymeric segment, as described below. In one embodiment, PS is a linear polymeric segment terminating with a methylene group at both ends of the segment; the segment is derived from a polymer selected from the group consisting of: polyethylene oxide, block or random copolymers of polyethylene oxide and polypropylene oxide, and triblock copolymers of polyethylene oxide and polypropylene oxide. As used herein “derived from a polymer” when referring to a polymeric segment, means that the polymeric segment has the structure of the polymer without the polymer's terminal end groups (e.g., hydroxyl groups), and therefore both ends of the polymeric segment are terminated with a methylene group.

R1 is at least one nucleophilic group capable of reacting with aldehyde groups to form at least one reversible covalent bond. Suitable R1 groups include, but are not limited to, primary amine, secondary amine, and carboxyhydrazide. R2 is at least one functional group which is not capable of reacting with an aldehyde, a primary amine, a secondary amine, or R1 to form a covalent bond such that the oligomer does not induce gelation when mixed in an aqueous solution with the oxidized polysaccharide (i.e., the oligomer does not function as a crosslinking agent). Suitable R2 groups include, but are not limited to, hydroxy, methoxy, ethoxy, propoxy, butoxy, and phenoxy. Suitable oligomers have a weight-average molecular weight of about 200 to about 4,000 Daltons, more particularly about 200 to about 3,000 Daltons, and more particularly about 350 to about 2,000 Daltons. The oligomer is water soluble.

Suitable oligomers are available commercially from companies such as Sigma-Aldrich (St. Louis, Mo.), or can be synthesized using methods known in the art. For example, a methoxy PEG amine may be prepared by mesylation of a suitable molecular weight methoxy PEG alcohol (available from Sigma-Aldrich), followed by amination of the mesylated intermediate, as described in detail in the Examples herein below. Additionally, various linking groups at the ends of the polymeric segment may be used to attach R1 and R2 to the polymeric segment. Nonlimiting examples of linking groups include S—R₂—CH₂, and NH—R₂—CH₂, wherein R₂ is an alkylene group having from 1 to 5 carbon atoms. For example, a suitable molecular weight methoxy PEG alcohol may be reacted with methanesulfonyl chloride in a suitable solvent, such as dichloromethane, in the presence of a base such as tripentylamine, to form the mesylate derivative, which is subsequently reacted with a diamine such as ethylene diamine to form an oligomer wherein R1 (a primary amine group) is attached through the linker NH—CH₂—CH₂, which is at one end of the polymeric segment (i.e., NH—CH₂—CH₂—R1).

In one embodiment, the oligomer is methoxy polyethylene glycol amine wherein PS is a linear polymeric segment derived from polyethylene oxide, R1 is a primary amine group and R2 is a methoxy group.

### Method of Dissolving an Oxidized Polysaccharide

The method of dissolving an oxidized polysaccharide in an aqueous solution comprises the following steps: a) providing at least one oxidized polysaccharide, as described above, in dry powder form; b) providing an aqueous solution; c) providing at least one oligomer of formula (1); d) combining (a), (b), and (c) in any order to form a heterogeneous mixture; and e) agitating the heterogeneous mixture to effect dissolution of the oxidized polysaccharide to form an aqueous solution of the oxidized polysaccharide.

To provide the oxidized polysaccharide in dry form, the oxidized polysaccharide product resulting from the oxidation of the polysaccharide is recovered and dried using methods known in the art, for example drying under vacuum or lyophilization. The oxidized polysaccharide is provided in an amount sufficient to give a concentration from about 5% to about 40% by weight, more particularly from about 5% to about 30% by weight, and more particularly from about 10% to about 30% by weight relative to the total weight of the final aqueous solution of the oxidized polysaccharide. A mixture of two or more different oxidized polysaccharides may also be used. For example, a mixture of oxidized polysaccharides having a different polysaccharide backbone, a different oxidation conversion, and/or a different average molecular weight, may be used. Where a mixture of different oxidized polysaccharides is used, the total amount of the oxidized polysaccharides is sufficient to give a concentration from about 5% to about 40% by weight, more particularly from about 5% to about 30% by weight, and more particularly from about 10% to about 30% by weight relative to the total weight of the final aqueous solution of the oxidized polysaccharide.

The aqueous solution comprises water and optionally, various additives, as described below. In one embodiment, the aqueous solution is water. Then, the oxidized polysaccharide, the aqueous solution, and the oligomer, as described above, are combined in any order to form the heterogeneous mixture. For example, the oxidized polysaccharide may be added to water, followed by the addition of the oligomer, or the oligomer may be added first to water, followed by the addition of the oligomer. Alternatively, a solution of the oligomer in water may be added to the aqueous solution before or after the addition of the oxidized polysaccharide. Then, the resulting heterogeneous mixture is agitated to effect the dissolution of the oxidized polysaccharide. The agitation may be accomplished using methods to known in the art, including, but not limited to, stirring, shaking, vortexing, and the like. A mixture of different oligomers having different polymeric segments (PS), different, R1 groups, different R2 groups, and/or different average molecular weights may also be used.

The amount of the oligomer additive necessary to provide the desired dissolution time depends on the oxidized polysaccharide used and on its concentration, and can be determined by one skilled in the art using routine experimentation. Useful oligomer concentrations are from about 0.5% to about 30% by weight, more particularly from about 1% to about 20% by weight, and more particularly from about 1% to about 10% by weight relative to the total weight of the final aqueous solution of the oxidized polysaccharide. If a mixture of oligomers is used, the total concentration of the oligomers is from about 0.5% to about 30% by weight, more particularly from about 1% to about 20% by weight, and more particularly from about 1% to about 10% by weight relative to the total weight of the final aqueous solution of the oxidized polysaccharide.

In one embodiment, the invention provides an aqueous composition comprising a) water; b) at least one oxidized polysaccharide, as described above; and c) at least one oligomer of formula (1).

### Hydrogel Tissue Adhesives

The aqueous solution of the oxidized polysaccharide described above may be used in combination with an aqueous solution comprising an amine-containing component to prepare hydrogel tissue adhesives and sealants for medical and veterinary applications, including, but not limited to, wound closure, supplementing or replacing sutures or staples in internal surgical procedures such as intestinal anastomosis and vascular anastomosis, tissue repair, ophthalmic procedures, drug delivery, and to prevent post-surgical adhesions. For example, the aqueous solution of the oxidized polysaccharide may be used in combination with an aqueous solution comprising a multi-arm polyether amine (Kodokian et al., copending and commonly owned U.S. Patent Application Publication No. 2006/0078536), and described in detail in the Examples herein below. Alternatively, the aqueous solution of the oxidized polysaccharide may be used in combination with an aqueous solution comprising a polymer having amino groups such as chitosan or a modified polyvinyl alcohol having amino groups (Goldmann, U.S. Patent Application Publication No. 2005/000289), or with an aqueous solution comprising an amino group containing polymer such as poly L-lysine (Nakajima et al., U.S. Patent Application Publication No. 2008/0319101).

The addition of the oligomer of formula (1) to the oxidized polysaccharide solution results in a decrease in the degradation time of a hydrogel formed by the combination of the oxidized polysaccharide and a multi-arm polyether amine, as shown in the Examples herein below. In general, the larger the amount of the oligomer used, the greater is the effect on reducing the degradation time of the hydrogel. The gelation time to form the hydrogel may also be increased at high concentrations of the oligomer.

For use as a component to prepare a hydrogel tissue adhesive or sealant, it is preferred that the aqueous solution of the oxidized polysaccharide be sterilized to prevent infection. Any suitable sterilization method known in the art that does not adversely affect the ability of the oxidized polysaccharide to react to form an effective hydrogel may be used, including, but not limited to, electron beam irradiation, gamma irradiation, ethylene oxide sterilization, or ultra-filtration through a 0.2 μm pore membrane.

The aqueous solution of the oxidized polysaccharide may comprise various additives depending on the intended application. Preferably, the additive does not interfere with effective gelation to form a hydrogel. The amount of the additive used depends on the particular application and may be readily determined by one skilled in the art using routine experimentation. For example, the aqueous solution of the oxidized polysaccharide may comprise at least one additive selected from pH modifiers, antimicrobials, colorants, surfactants, pharmaceutical drugs and therapeutic agents.

The aqueous solution of the oxidized polysaccharide may optionally include at least one pH modifier to adjust the pH of the solution. Suitable pH modifiers are well known in the art. The pH modifier may be an acidic or basic compound. Examples of acidic pH modifiers include, but are not limited to, carboxylic acids, inorganic acids, and sulfonic acids. Examples of basic pH modifiers include, but are not limited to, hydroxides, alkoxides, nitrogen-containing compounds other than primary and secondary amines, and basic carbonates and phosphates.

The aqueous solution of the oxidized polysaccharide may optionally include at least one antimicrobial agent. Suitable antimicrobial preservatives are well known in the art. Examples of suitable antimicrobials include, but are not limited to, alkyl parabens, such as methylparaben, ethylparaben, propylparaben, and butylparaben; triclosan; chlorhexidine; cresol; chlorocresol; hydroquinone; sodium benzoate; and potassium benzoate.

The aqueous solution of the oxidized polysaccharide may optionally include at least one colorant to enhance the visibility of the solution. Suitable colorants include dyes, pigments, and natural coloring agents. Examples of suitable colorants include, but are not limited to, FD&C and D&C colorants, such as FD&C Violet No. 2, FD&C Blue No. 1, D&C Green No. 6, D&C Green No. 5, D&C Violet No. 2; and natural colorants such as beetroot red, canthaxanthin, chlorophyll, eosin, saffron, and carmine.

The aqueous solution of the oxidized polysaccharide may optionally include at least one surfactant. Surfactant, as used herein, refers to a compound that lowers the surface tension of water. The surfactant may be an ionic surfactant, such as sodium lauryl sulfate, or a neutral surfactant, such as polyoxyethylene ethers, polyoxyethylene esters, and polyoxyethylene sorbitan.

Additionally, the aqueous solution of the oxidized polysaccharide may optionally include at least one pharmaceutical drug or therapeutic agent. Suitable drugs and therapeutic agents are well known in the art (for example see the United States Pharmacopeia (USP), Physician's Desk Reference (Thomson Publishing), The Merck Manual of Diagnosis and Therapy 18th ed., Mark H. Beers and Robert Berkow (eds.), Merck Publishing Group, 2006; or, in the case of animals, The Merck Veterinary Manual, 9th ed., Kahn, C. A. (ed.), Merck Publishing Group, 2005). Nonlimiting examples include, but are not limited to, anti-inflammatory agents, for example, glucocorticoids such as prednisone, dexamethasone, budesonide; non-steroidal anti-inflammatory agents such as indomethacin, salicylic acid acetate, ibuprofen, sulindac, piroxicam, and naproxen; fibrinolytic agents such as a tissue plasminogen activator and streptokinase; anti-coagulants such as heparin, hirudin, ancrod, dicumarol, sincumar, iloprost, L-arginine, dipyramidole and other platelet function inhibitors; antibodies; nucleic acids; peptides; hormones; growth factors; cytokines; chemokines; clotting factors; endogenous clotting inhibitors; antibacterial agents; antiviral agents; antifungal agents; anti-cancer agents; cell adhesion inhibitors; healing promoters; vaccines; thrombogenic agents, such as thrombin, fibrinogen, homocysteine, and estramustine; radio-opaque compounds, such as barium sulfate and gold particles and radiolabels.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Reagents

Methoxy PEG amines (CAS No. 80506-64-5) of several average molecular weights (i.e., 5000, 2000, and 750 Da) were obtained from Sigma-Aldrich. A methoxy PEG amine having an average molecular weight of 350 Da was synthesized as described below. A methoxy PEG amine having an average molecular weight of 750 Da was also synthesized using the same procedure. The methoxy PEG amine having an average molecular weight of 750 Da that was obtained from Sigma-Aldrich was used in the following Examples, except where use of the synthesized material is specifically indicated. In the following Examples, methoxy PEG amines are referred to as “MPA” followed by the average molecular weight. For example, MPA 2000 is a methoxy PEG amine having an average molecular weight 2000 Da.

### Preparation of MPA 350

A 350 molecular weight methoxy PEG amine was synthesized using a two-step procedure involving mesylation of a similar molecular weight methoxy PEG alcohol, followed by amination of the mesylated intermediate.

Step 1—Mesylation of Methoxy PEG Alcohol:

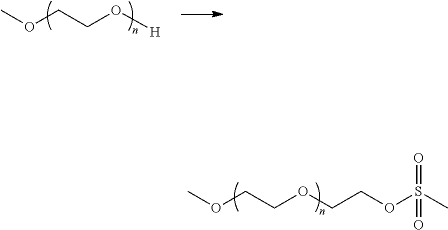

In the first step, 17.502 g (0.05 mol) of methoxy PEG alcohol having an average molecular weight of 350 Da (Sigma-Aldrich) was dissolved in 250 mL of methylene chloride at room temperature (RT) in a 500 mL, 3-neck, round-bottom flask. To this solution was added 13.94 mL (0.1 mol) of triethylamine, followed by the dropwise addition of 7.74 mL (0.1 mol) of methanesulfonyl chloride (fuming, slight exotherm). The resulting reaction solution was stirred overnight at RT while maintaining a nitrogen blanket. Then, the reaction solution was diluted with 250 mL of chloroform and washed with 1.0 M potassium hydrogen phosphate (2×100 mL), 1.0 M potassium carbonate (2 x 100 mL), and deionized water (3×100 mL). The organic layer was dried over magnesium sulfate, filtered, and concentrated using a rotary evaporator to produce an amber oil product. The amber oil product was dried under high vacuum overnight (i.e., less than 100 mTorr (13.3 Pa)). The final weight of the dried product was 20.32 g. The identity of the product was confirmed by ¹H NMR in deuterated chloroform.

### Step 2—Amination of Mesylation Product:

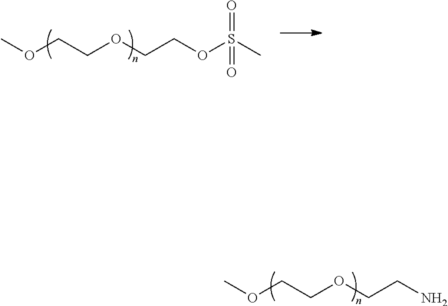

In the second step, 19.8 g of the mesylated product from step 1 was dissolved in 400 mL of ammonium hydroxide solution (28-30% in water) in a tightly capped bottle and stirred at RT for 5 days. The solution was then sparged with nitrogen for 6 hours to drive off residual ammonia (to approximately 85% of the original volume). The resulting solution was diluted with 200 mL of 2.0 M potassium carbonate solution and extracted with chloroform (3×150 mL). The chloroform layers were combined, dried over magnesium sulfate, and concentrated using a rotary evaporator to produce a pale yellow oil. The oil was dried further under high vacuum (i.e., less than 90 mTorr (12.0 Pa)). The final weight of the resulting product was 14.4 g. The identity of the product was confirmed by ¹H NMR in deuterated dimethyl sulfoxide.

### Preparation of MPA 750

A 750 molecular weight methoxy PEG amine was synthesized using the two-step procedure described above for the preparation of MPA 350. The starting methoxy PEG alcohol having an average molecular weight of about 750 Daltons was obtained from Sigma-Aldrich.

### Preparation of Dextran Aldehyde (D10-50)

Dextran aldehyde is made by oxidizing dextran in aqueous solution with sodium metaperiodate. An oxidized dextran with about 50% oxidation conversion (i.e., about half of the glucose rings in the dextran polymer are oxidized to dialdehydes) is prepared from dextran having a weight-average molecular weight of 8,500 to 11,500 Daltons (Sigma) by the method described by Cohen et al. (copending and commonly owned International Patent Application Publication No. WO 2008/133847). A typical procedure is described here.

A 20-L reactor equipped with a mechanical stirrer, addition funnel, internal temperature probe, and nitrogen purge is charged with 1000 g of the dextran and 9.00 L of de-ionized water. The mixture is stirred at ambient temperature to dissolve the dextran and then cooled to 10 to 15° C. To the cooled dextran solution is added over a period of an hour, while keeping the reaction temperature below 25° C., a solution of 1000 g of sodium periodate dissolved in 9.00 L of de-ionized water. Once all the sodium periodate solution has been added, the mixture is stirred at 20 to 25° C. for 4 more hours. The reaction mixture is then cooled to 0° C. and filtered to clarify. Calcium chloride (500 g) is added to the filtrate, and the mixture is stirred at ambient temperature for 30 min and then filtered. Potassium iodide (400 g) is added to the filtrate, and the mixture is stirred at ambient temperature for 30 min. A 3-L portion of the resulting red solution is added to 9.0 L of acetone over a period of 10 to 15 min with vigorous stirring by a mechanical stirrer during the addition. After a few more minutes of stirring, the agglomerated product is separated from the supernatant liquid. The remaining red solution obtained by addition of potassium iodide to the second filtrate is treated in the same manner as above. The combined agglomerated product is broken up into pieces, combined with 2 L of methanol in a large stainless steel blender, and blended until the solid becomes granular. The granular solid is recovered by filtration and dried under vacuum with a nitrogen purge. The granular solid is then hammer milled to a fine powder. A 20-L reactor is charged with 10.8 L of de-ionized water and 7.2 L of methanol, and the mixture is cooled to 0° C. The granular solid formed by the previous step is added to the reactor and the slurry is stirred vigorously for one hour. Stirring is discontinued, and the solid is allowed to settle to the bottom of the reactor. The supernatant liquid is decanted by vacuum, 15 L of methanol is added to the reactor, and the slurry is stirred for 30 to 45 min while cooling to 0° C. The slurry is filtered in portions, and the recovered solids are washed with methanol, combined, and dried under vacuum with a nitrogen purge to give about 600 g of the oxidized dextran, which is referred to herein as D10-50.

The degree of oxidation of the product is determined by proton NMR to be about 50% (equivalent weight per aldehyde group=146). In the NMR method, the integrals for two ranges of peaks are determined, specifically, —O₂CHx- at about 6.2 parts per million (ppm) to about 4.15 ppm (minus the HOD peak) and —OCHx- at about 4.15 ppm to about 2.8 ppm (minus any methanol peak if present). The calculation of oxidation level is based on the calculated ratio (R) for these areas, specifically, R═(OCH)/(O₂CH).

### Preparation of Eight-Arm PEG 10K Octaamine (P8-10-1):

Eight-arm PEG 10K octaamine (Mₙ=10 kDa) is synthesized using the two-step procedure described by Chenault in co-pending and commonly owned U.S. Patent Application Publication No. 2007/0249870. In the first step, the 8-arm PEG 10K chloride is made by reaction of thionyl chloride with the 8-arm PEG 10K octaalcohol. In the second step, the 8-arm PEG 10K chloride is reacted with aqueous ammonia to yield the 8-arm PEG 10K octaamine. A typical procedure is described here.

The 8-arm PEG 10K octaalcohol (Mₙ=10000; SunBright HGEO-10000; NOF Corp.), (100 g in a 500-mL round-bottom flask) is dried either by heating with stirring at 85° C. under vacuum (0.06 mm of mercury (8.0 Pa)) for 4 hours or by azeotropic distillation with 50 g of toluene under reduced pressure (2 kPa) with a pot temperature of 60° C. The 8-arm PEG 10K octaalcohol is allowed to cool to room temperature and thionyl chloride (35 mL, 0.48 mol) is added to the flask, which is equipped with a reflux condenser, and the mixture is heated at 85° C. with stirring under a blanket of nitrogen for 24 hours. Excess thionyl chloride is removed by rotary evaporation (bath temp 40° C.). Two successive 50-mL portions of toluene are added and evaporated under reduced pressure (2 kPa, bath temperature 60° C.) to complete the removal of thionyl chloride. Proton NMR results from one synthesis are:

¹H NMR (500 MHz, DMSO-d6) δ 3.71-3.69 (m, 16H), 3.67-3.65 (m, 16H), 3.50 (s, ˜800H).

The 8-arm PEG 10K octachloride (100 g) is dissolved in 640 mL of concentrated aqueous ammonia (28 wt %) and heated in a pressure vessel at 60° C. for 48 hours. The solution is sparged for 1-2 hours with dry nitrogen to drive off 50 to 70 g of ammonia. The solution is then passed through a column (500 mL bed volume) of strongly basic anion exchange resin (Purolite® A-860, The Purolite Co., Bala-Cynwyd, Pa.) in the hydroxide form. The eluant is collected and three 250-mL portions of de-ionized water are passed through the column and also collected. The aqueous solutions are combined, concentrated under reduced pressure (2 kPa, bath temperature 60° C.) to about 200 g, frozen in portions and lyophilized to give the 8-arm PEG 10K octaamine, referred to herein as P8-10-1, as a colorless waxy solid.

### General Methods

### Preparation of Hydrogel Precursor Solutions

Oxidized dextran solutions and multi-arm PEG amine solutions were prepared by dissolving the desired amount of oxidized dextran or multi-arm PEG amine in distilled water to achieve the desired concentration (weight %). The multi-arm PEG amine typically dissolved readily at room temperature. In the absence of additives, the oxidized dextran dissolved slowly at room temperature, but dissolved completely after heating at 37° C. overnight.

Various methoxy PEG amines were added to either the oxidized dextran or multi-arm PEG amine solutions, or both. A formulation with an additive was designed by removing a quantity of water from a control formulation and replacing it with the same quantity of the additive. Specific procedures for introducing additives are described in the Examples below.

### Gelation Time Measurements

The gelation time upon mixing the hydrogel precursor solutions was studied to assess the ease of application for in vivo use. The oxidized dextran solution (0.10 mL) was placed in a vial. Then, 0.10 mL of the multi-arm PEG amine solution was added to the vial and the mixture was immediately stirred with a small spatula until the mixture gelled to the point where it held its shape without flowing. This time was measured and taken as the gelation time.

### Degradation Time Measurements

The degradation behavior of hydrogels at 37° C. in Dulbecco's phosphate buffered saline at pH 7.4 (DPBS, 1× without calcium or magnesium, Invitrogen, Carlsbad, Calif.; cat. 14190 or Mediatech, Herndon, Va.; cat. 21-031) was studied as follows to assess acceptability of the hydrogel formulation for in vivo use. A double-barrel syringe (1:1 v/v) with a 16-step static mixing tip was used to prepare a hydrogel test strip. The oxidized dextran solution was added to one side of the double-barrel syringe, and the multi-arm PEG amine solution was added to the other side. The mixing tip was cut 5 mm from the end to make a larger exit diameter.

A hydrogel formulation was cast using the double-barrel syringe with mixing tip into a 1 mm thick by 6.8 mm wide by approximately 70 mm long mold. After 15 min, the ends were trimmed and the resulting hydrogel strip was cut into 2 test strips, each 30 mm x 6.8 mm x 1 mm in size. After weighing, the strips were each placed in a 20 mL vial containing DPBS buffer. The vials were capped and placed in an incubator shaker at 37° C. and 80 rpm. The hydrogel test strips were typically weighed at 2 hours and 5 hours on the first day, and every 24 hours thereafter until the weight of the test strip was less than 50% of its initial weight. At each time, the gel strip was removed from buffer, drained of excess liquid, and weighed. The strip was then placed in a vial with fresh DPBS and returned to the incubator.

This procedure resulted in a plot of gel weight versus time, expressed as percent of initial weight versus time. Typically, there was an initial increase in weight due to equilibrium swelling, followed by some additional swelling as crosslinks are broken and finally a loss of weight as soluble degradation products diffuse from the gel. Fragments of the gel may linger for some time. The time to 50% of the initial weight was used as a meaningful parameter of the degradation curve for comparing formulations.

This time, referred to herein as the degradation time, was estimated by interpolation between the time point at which the weight is just above 50% and the time point at which the weight is just below 50%. Reported values are averages of determinations on the two gel strip samples.

### Examples 1-4

### Effect of Methoxy PEG Amines of Different Molecular Weight on Dissolution of Oxidized Dextran

The purpose of these Examples was to demonstrate the effect of methoxy PEG amines on the dissolution rate of oxidized dextran.

Methoxy PEG amines having average molecular weights of 750, 2000, and 5000 Da (obtained from Sigma-Aldrich) were each dissolved in deionized water in a vial. Then, oxidized dextran D10-50 powder was poured into the vial all at once. The vial was capped and then stirred with a magnetic stirrer at RT. For comparison, the same amount of D10-50 was poured into a vial with deionized water without the methoxy PEG amine (Example 4, Comparative). The compositions and observations are summarized in Table 1.

TABLE 1
Effect of Methoxy PEG amine on Dissolution
Rate of Oxidized Dextran

| | MPA Molecular | MPA | D10-50 | Dissolution | |
|---|---|---|---|---|---|
| Example | Weight (Da) | (wt %) | (wt %) | Time | |
| 1 | 750 | 8% | 8% | ≦5 | min |
| 2 | 2000 | 8% | 8% | 5-10 | min |
| 3 | 5000 | 8% | 8% | >24 | hours |
| 4 Compar- | none | 0% | 8% | >24 | hours |
| ative | | | | | |

The results in Table 1 suggest that in compositions containing 8 wt % MPA 750 (Example 1) and MPA 2000 (Example 2) the oxidized dextran dissolved completely at room temperature in just a few minutes. In a composition containing MPA 5000 (Example 3) and the comparative formulation without MPA (Example 4, Comparative), the oxidized dextran did not dissolve fully even after 24 hours. Those compositions required a few hours in an incubator at 37° C. to effect complete dissolution of the oxidized dextran.

### Examples 5-8

### Gelation Times for the Formation of Hydrogels from Oxidized Dextran and a Multi-Arm PEG Amine in the Presence of Methoxy PEG Amines

The purpose of these Examples was to demonstrate the formation of hydrogels from an oxidized dextran (D10-50) and a multi-arm PEG amine (P8-10-1) in the presence of a methoxy PEG amine additive. The time required to form the hydrogel (i.e., the gelation time) was also determined.

Hydrogels were formed by mixing an aqueous solution containing an oxidized dextran (i.e., D10-50) containing a methoxy PEG amine with an aqueous solution containing a multi-arm PEG amine (i.e., P8-10-1) using the method described above in General Methods. The oxidized dextran solutions used are described in Examples 1-4. The results are summarized in Table 2.

TABLE 2
Gelation Times for the Formation of Hydrogels

| | Oxidized Dextran | P8-10-1 | Gelation Time |
|---|---|---|---|
| Example | Solution | (wt %) | (sec) |
| 5 | Example 1 | 30% | 90-120 |
| 6 | Example 2 | 30% | 25-30 |
| 7 | Example 3 | 30% | 8-12 |
| 8 Compar- | Example 4 | 30% | 8-12 |
| ative | Comparative | | |

The results given in Table 2 suggest that the lower molecular weight MPA additives that dramatically enhance dissolution of dextran-aldehyde (Examples 5 and 6) also significantly retard gelation time compared to the comparative Example without the methoxy PEG amine additive (Example 8, Comparative).

### Examples 9-12

### Effect of Different Concentrations of Methoxy PEG Amine 750 on the Dissolution of Oxidized Dextran

Various concentrations of MPA 750 were each dissolved in deionized water in a vial. Then oxidized dextran D10-50 powder was poured into the vial all at once. The vial was capped and then stirred with a magnetic stirrer at room temperature until the D10-50 was dissolved. The compositions and observations are summarized in Table 3.

TABLE 3
Effect of MPA 750 on Dissolution Rate of Oxidized Dextran

| | MPA 750 | D10-50 | Dissolution Time |
|---|---|---|---|
| Example | (wt %) | (wt %) | (min) |
| 9 | 8% | 8% | 1-2 |
| 10 | 4% | 8% | 1-2 |
| 11 | 2% | 8% | 2-3 |
| 12 | 1% | 8% | 5 (slight remaining |
| | | | particulate) |

The results shown in Table 3 suggest that only 1 or 2 wt % of MPA 750 (Examples 11 and 12) enhances the dissolution rate of D10-50 (see Example 4, Comparative).

### Examples 13-16

### Gelation Times for the Formation of Hydrogels from Oxidized Dextran and a Multi-Arm PEG Amine in the Presence of MPA 750

The purpose of these Examples was to demonstrate the formation of hydrogels from an oxidized dextran (D10-50) and a multi-arm PEG amine (P8-10-1) in the presence of MPA 750 at different concentrations. The time required to form the hydrogel (i.e., the gelation time) was also determined.

Hydrogels were formed by mixing an aqueous solution containing an oxidized dextran (i.e., D10-50) containing MPA 750 with an aqueous solution containing a multi-arm PEG amine (i.e., P8-10-1) using the method described above in General Methods. The oxidized dextran solutions used are described in Examples 9-12. The results are summarized in Table 4.

TABLE 4
Gelation Times for the Formation of Hydrogels

| | Oxidized Dextran | P8-10-1 | Gelation Time |
|---|---|---|---|
| Example | Solution | (wt %) | (sec) |
| 13 | Example 9 | 30% | 48-58 |
| 14 | Example 10 | 30% | 22-27 |
| 15 | Example 11 | 30% | 13-18 |
| 16 | Example 12 | 30% | 10-14 |

The data in Table 4 suggest that at the lower MPA 750 concentrations, i.e., 2 wt % (Example 15) and 1 wt % (Example 16), the effect on gelation time is fairly minor (see Example 8, Comparative). At 2 wt % MPA 750 and 8 wt % D10-50, complete reaction of the amines on MPA 750 only represents about 5% of the available aldehydes on D10-50. Therefore, 95% of the aldehyde groups of D10-50 would still be available to crosslink when combined with the P8-10-1 multi-arm PEG amine.

### Examples 17-21

### Effect of Different Concentrations of Methoxy PEG Amine 750 on the Dissolution of High Concentrations of Oxidized Dextran

Various concentrations of MPA 750 were each dissolved in deionized water in a vial. Then oxidized dextran D10-50 powder was poured into the vial all at once. The vial was capped and then stirred with a magnetic stirrer at room temperature until the D10-50 was dissolved. The compositions and observations are summarized in Table 5.

TABLE 5
Effect of MPA 750 on Dissolution Rate of Oxidized Dextran

| | MPA | D10- | | | Dissolu- |
|---|---|---|---|---|---|
| | 750 | 50 | | | tion Time, |
| | (wt | (wt | Dissolution Time, | | Complete |
| Example | %) | %) | Partial | | (hours) |
| 17 | 20% | 25% | 5 min | (some dissolved) | >72 |
| 18 | 10% | 25% | 10 min | (some dissolved) | 72 |
| 19 | 5% | 25% | 10 min | (most dissolved) | 2.5 |
| 20 | 2.5% | 25% | 5 min | (most dissolved) | 4 |
| 21 Compar- | 0% | 25% | 4.5 hours | (gelatinous) | 72 |
| ative | | | | | |

The results shown in Table 5 suggest that, although the addition of MPA 750 does not result in complete dissolution of 25 wt % D10-50 in minutes, its effect is still dramatic. In the absence of MPA 750 (Example 21, Comparative), the mixture of D10-50 and water is an unstirrable solid for several hours until it slowly becomes gelatinous. By contrast, the addition of MPA 750 enables the mixture to quickly become flowable and for part or most of the D10-50 to dissolve in minutes.

### Examples 22-26

### Gelation Times for the Formation of Hydrogels from Oxidized Dextran at High Concentration and a Multi-Arm PEG Amine in the Presence of Different Concentrations MPA 750

The purpose of these Examples was to demonstrate the formation of hydrogels from an oxidized dextran (D10-50) at high concentration and a multi-arm PEG amine (P8-10-1) in the presence of MPA 750 at different concentrations. The time required to form the hydrogel (i.e., the gelation time) was also determined.

Hydrogels were formed by mixing an aqueous solution containing a high concentration of oxidized dextran (i.e., D10-50) containing MPA 750 with an aqueous solution containing a multi-arm PEG amine (i.e., P8-10-1) using the method described above in General Methods. The oxidized dextran solutions used are described in Examples 17-21. The results are summarized in Table 6.

TABLE 6
Gelation Times for the Formation of Hydrogels

| | Oxidized Dextran | P8-10-1 | Gelation Time |
|---|---|---|---|
| Example | Solution | (wt %) | (sec) |
| 22 | Example 17 | 30% | 40-50 |
| 23 | Example 18 | 30% | 15-20 |
| 24 | Example 19 | 30% | 6-12 |
| 25 | Example 20 | 30% | 5-10 |
| 26 Compar- | Example 21 | 30% | 5-8 |
| ative | Comparative | | |

The data in Table 6 suggest that at the lower MPA 750 concentrations, i.e., 5 wt % (Example 24) and 2.5 wt % (Example 25), the effect on gelation time is fairly minor as the gelation times are comparable to the gelation time in the absence of MPA 750 (Example 26, Comparative).

### Examples 27-32

### Effect of Methoxy PEG Amines Having Different Molecular Weight on In Vitro Degradation Time of Hydrogels

The effect of methoxy PEG amine addition was studied using a base formulation of 12% D10-50 oxidized dextran in aqueous solution and 40% P8-10-1 multi-arm PEG amine in aqueous solution. Formulations were prepared incorporating various amounts of methoxy PEG amine of 750, 2000, or 5000 average molecular weight in place of water in the oxidized dextran solution. The degradation time of the resulting hydrogels was determined as described in General Methods. The formulations and degradation times are shown in Table 7.

TABLE 7
In Vitro Degradation Time Of Hydrogels

| | | | | | Degradation |
|---|---|---|---|---|---|
| | D10-50 | P8-10-1 | MPA MW | MPA | Time |
| Example | (wt %) | (wt %) | (Da) | (wt %) | (hours) |
| 27 Compar- | 12% | 40% | none | 0% | 151 |
| ative | | | | | |
| 28 | 12% | 40% | 750 | 1% | 88 |
| 29 | 12% | 40% | 2000 | 2% | 113 |
| 30 | 12% | 40% | 2000 | 4% | 36 |
| 31 | 12% | 40% | 5000 | 5% | 47 |
| 32 | 12% | 40% | 5000 | 10% | 26 |

The results shown in Table 7 suggest that the addition of methoxy PEG amine promotes degradation of the hydrogels compared with the same formulation without methoxy PEG amine (Example 27, Comparative). Although the addition of all of the methoxy PEG amines reduced degradation time, the lower molecular weight methoxy PEG amines had a greater effect at lower concentrations. For example, only 1% of MPA 750 reduced the degradation time from 151 to 88 hours (compare Example 27 with Example 28), while 2% of MPA 2000 reduced the degradation time from 151 to 113 hours (compare Example 27 with Example 29).

### Examples 33-39

### Effect of Methoxy PEG Amines on Gelation Time and In Vitro Degradation Time

The effect of methoxy PEG amine addition on gelation time and in vitro degradation time was studied using base formulations of 8 wt % and 10 wt % D10-50 oxidized dextran in aqueous solution and 30 wt % P8-10-1 multi-arm PEG amine in aqueous solution. Formulations were prepared incorporating various amounts of methoxy PEG amine of 350 or 750 molecular weight in place of water in the oxidized dextran solution. The gelation times and in vitro degradation times were determined as described in General Methods. The formulations and results are shown in Table 8.

TABLE 8
Gelation Times and In Vitro Degradation Times of Hydrogels

| | | | | | | Degra- |
|---|---|---|---|---|---|---|
| | | | MPA | | Gelation | dation |
| | D10-50 | P8-10-1 | MW | MPA | Time | Time |
| Example | (wt %) | (wt %) | (Da) | (wt %) | (sec) | (hours) |
| 33 | 8% | 30% | none | 0% | 7-10 | 26 |
| Comparative | | | | | | |
| 34 | 8% | 30% | 350 | 1% | 8-16 | 4 |
| 35 | 8% | 30% | 750 | 2% | 9-15 | 4 |
| 36 | 10% | 30% | none | 0% | 4-8 | 85 |
| Comparative | | | | | | |
| 37 | 10% | 30% | 350 | 1.5% | 7-14 | 4 |
| 38 | 10% | 30% | 750 | 2% | 7-12 | 18 |
| 39 | 10% | 30% | 750 | 3% | 7-14 | 4 |

The results shown in Table 8 suggest that at these low levels of methoxy PEG amine additive, the effect on gelation time is minor. However, the shortening of degradation time is dramatic. For example, addition of only 1 wt % MPA 350 or 2 wt % MPA 750 reduces degradation time from 26 hours to 4 hours for the base formulation with 8 wt % D10-50 (Examples 34 and 35 compared to Example 33, Comparative). Similar large effects are seen when MPA 350 or 750 is added to the base formulation with 10 wt % D10-50 (Examples 37, 38, and 39 compared to Example 36, Comparative).

### Example 40

### Effect of Methoxy PEG Amine Added to Multi-Arm PEG Amine Solution on Gelation Time and In Vitro Degradation Time

To compare the effect of adding methoxy PEG amine to the multi-arm PEG amine solution with the effect of adding it to the oxidized dextran solution, the formulation of Example 39 was repeated, except that the 3% MPA 750 was added to the multi-arm PEG amine solution, replacing an equal amount of water. The gelation time and in vitro degradation time were determined using the same methods used for Examples 33 through 39. The gelation time of this formulation was 4-8 sec and the degradation time was 40 hours.

Comparison of these results with those for Example 36, Comparative and Example 39 illustrates the significant influence of the manner of addition of the MPA. The degradation time was reduced from 85 to 40 hours when 3 wt % MPA 750 was added to the P8-10-1 solution (Example 36, Comparative versus Example 40). But the reduction was from 85 to 4 hours when the same amount of MPA 750 was instead added to the D10-50 solution (Example 36, Comparative versus Example 39). Gelation time was not measurably affected by the addition of 3 wt % MPA 750 to the P8-10-1 solution, unlike the modest effect on gelation time observed when 3 wt % MPA 750 was added to the D10-50 solution. Therefore, these results demonstrate that adding the methoxy PEG amine to the oxidized dextran solution has a larger effect on degradation time than adding it to the multi-arm PEG amine solution.

### Examples 41 and 42

### Cytotoxicity Testing of Methoxy PEG Amines

The purpose of these Examples was to demonstrate the safety of methoxy PEG amines in an in vitro cytotoxicity test.

Methoxy PEG amine solutions (1.0 wt %) were prepared and tested for cytotoxicity. MPA 750 (102.2 mg) from Sigma-Aldrich (Example 41) and MPA 750 (103.3 mg) synthesized as described in Reagents (Example 42) were placed in Falcon™ test tubes. Ten milliliters of Dulbecco's modified essential medium (DMEM) was added to each tube to give a 10 mg/mL working solution concentration. After the MPA dissolved in the cell culture medium, both media turned bright purple, indicating that MPA is responsible for an increase in pH. Both MPA solutions were transferred to a cell culture flask and incubated at 37° C. under 5% CO₂ in an incubator for at least one hour to allow the pH of the media to equilibrate to neutral pH. Both MPA solutions were filtered through a 0.22 μm filter unit before applying to the cells.

NIH 3T3 P20 cells were detached from the walls of a flask with the aid of trypsin and re-suspended at a suitable cell concentration of about a half million cells per well of a six well plate for samples, positive and negative control. To the positive control well was added 100 μl of Tween® 20 mixed with the cells. The negative control well cells were cultured with DMEM culture medium. The cells were imaged using a light microscope after 20 hours and 48 hours for extended toxicity evaluation. Both samples showed no toxicity for cells. Cell growth was the same as for the negative control. Therefore, 1% MPA 750, whether from Sigma-Aldrich (Example 41) or synthesized in the lab (Example 42), showed no toxicity to NIH 3T3 P20 cells, which suggests that the methoxy PEG amines are safe as an additive to hydrogels for use in the body.

### Examples 43-45

### Cytotoxicity Testing of Hydrogels Containing Methoxy PEG Amines

The purpose of these Examples was to demonstrate the safety of hydrogels containing methoxy PEG amines in an in vitro cytotoxicity test.

Hydrogels were prepared by dispensing precursor solutions, as shown in Table 9, from a double-barrel syringe through a 16-step mixing tip into a 0.45 mm thickness mold. The resulting gelled samples were cut into round disks with a weight range of 30-35 mg. The disks were placed into the wells of a six-well plate. All tools employed in the hydrogel formation were cleaned with 70% ethanol prior to use to minimize contamination.

TABLE 9
Precursor Solutions for Preparation of Hydrogels

| | D10-50 | P8-10-1 | MPA 750 |
|---|---|---|---|
| Example | (wt %) | (wt %) | (wt %) |
| 43, Compar- | 12% | 40% | none |
| ative | | | |
| 44 | 12% | 40% | 1% (from |
| | | | Sigma-Aldrich) |
| 45 | 12% | 40% | 1% (synthe- |
| | | | sized) |

NIH 3T3 P20 cells were detached from the walls of a flask with the aid of trypsin and re-suspended at a suitable cell concentration of about half million cells per well of a six well plate for samples, positive and negative control. To the positive control well was added 100 μl of Tween® 20 mixed with cells. The negative control well cells were cultured with DMEM culture medium. The cells were imaged using a light microscope after 20 hours and 48 hours for extended toxicity evaluation. All three samples showed no toxicity for cells. Cell growth was the same as for the negative control. For Examples 44 and 45, cells grew nicely even near the hydrogels, even better than for Example 43, Comparative without MPA 750. Therefore, hydrogels with MPA 750, whether from Sigma-Aldrich (Example 44) or synthesized in the lab (Example 45), show no toxicity to NIH 3T3 P20 cells, which suggests that the hydrogels containing methoxy PEG amines are safe for use in the body.

### Examples 46-51

### Burst Strength Testing of Hydrogel Formulations Containing Methoxy PEG Amine

The purpose of these Examples was to demonstrate the burst strength of a seal made with hydrogels containing a methoxy PEG amine additive of an incision made in swine uterine horn.

A 5 to 6-mm incision was made using a #15 surgical blade in a 6 to 8-cm section of clean, fresh swine uterine horn. The wound was sealed by applying a hydrogel formulation using a double-barrel syringe with a mixing tip at a thickness of 1-2 mm. After the hydrogel had been allowed to cure (typically 2-3 min), one end of the section of uterine horn was secured to a metal nipple with a nylon cable tie, and the other end was clamped shut. The metal nipple was connected by plastic tubing to a syringe pump equipped with a pressure meter. The section of uterine horn was submerged in a beaker of water, and purple dyed water was pumped by the syringe pump into the section at 11 mL/min. The pressure at which the sealed wound leaked was noted and recorded as the burst strength. Reported values are typically averages of 3 to 4 measurements. The burst strengths of several hydrogel formulations containing MPA 750 were determined. The formulations and results, given as the mean and standard deviation, are summarized in Table 10.

TABLE 10
Burst Strength of Hydrogels Containing Methoxy PEG Amine

| | D10-50 | P8-10-1 | MPA 750 | Burst Strength |
|---|---|---|---|---|
| Example | (wt %) | (wt %) | (wt %) | (psi) |
| 46 Compar- | 12% | 30% | none | 2.46 ± 0.04 |
| ative | | | | (17.0 ± 0.3 kPa) |
| 47 | 12% | 30% | 0.75% | 1.33 ± 0.35 |
| | | | | (9.17 ± 2 kPa) |
| 48 | 12% | 30% | 1.5% | 1.63 ± 0.2 |
| | | | | (11.2 ± 1 kPa) |
| 49 Compar- | 12% | 40% | none | 2.95 ± 1.17 |
| ative | | | | (20.3 ± 8.1 kPa) |
| 50 | 12% | 40% | 0.75% | 2.49 ± 0.6 |
| | | | | (17.2 ± 4 kPa) |
| 51 | 12% | 40% | 1.5% | 3.09 ± 0.31 |
| | | | | (21.3 ± 2 kPa) |

The results shown in Table 10 suggest that formulations containing MPA 750 at levels that enhance dissolution of oxidized dextran and promote degradation also exhibit burst strength that is adequate for adhesive and other in vivo applications.

1. A method of dissolving an oxidized polysaccharide in an aqueous solution, the method comprising the steps of:
a) providing at least one oxidized polysaccharide in dry powder form, said oxidized polysaccharide containing aldehyde groups, and having a weight-average molecular weight of about 1,000 to about 1,000,000 Daltons, and an equivalent weight per aldehyde group of about 65 to about 1500 Daltons;
b) providing an aqueous solution;
c) providing at least one oligomer of the formula: R1-(PS)—R2 wherein:
(i) PS is a linear polymeric segment comprising ethylene oxide monomers or a combination of ethylene oxide and propylene oxide monomers, wherein said ethylene oxide monomers comprise at least about 50 weight percent of said polymeric segment;
(ii) R1 is at least one nucleophilic group capable of reacting with aldehyde groups to form at least one reversible covalent bond;
(iii) R2 is at least one functional group which is not capable of reacting with an aldehyde, a primary amine, a secondary amine, or R1 to form a covalent bond, such that said oligomer does not induce gelation when mixed in the aqueous solution with (a);
(iv) said oligomer has a weight-average molecular weight of about 200 to about 4,000 Daltons; and
(v) said oligomer is water soluble;
d) combining (a), (b), and (c) in any order to form a heterogeneous mixture; and
e) agitating the mixture obtained in step (d) to effect dissolution of the oxidized polysaccharide to obtain an aqueous solution of the oxidized polysaccharide.

2. The method according to claim 1 wherein the oxidized polysaccharide is selected from the group consisting of oxidized derivatives of: dextran, carboxymethyldextran, starch, agar, cellulose, hydroxyethylcellulose, carboxymethylcellulose, pullulan, inulin, levan, and hyaluronic acid.

3. The method according to claim 2 wherein the oxidized polysaccharide is oxidized dextran.

4. The method according to claim 1 wherein the oligomer is methoxy polyethylene glycol amine wherein PS is a linear polymeric segment derived from polyethylene oxide, R1 is a primary amine, and R2 is methoxy.

5. The method according to claim 1 wherein the oxidized polysaccharide is provided in an amount sufficient to give a concentration of said oxidized polysaccharide from about 5% to about 40% by weight relative to the total weight of the aqueous solution obtained in step (e).

6. The method according to claim 1 wherein the oligomer is provided in an amount sufficient to give a concentration of said oligomer from about 0.5% to about 30% by weight relative to the total weight of the aqueous solution obtained in step (e).

7. The method according to claim 1 wherein R1 is selected from the group consisting of: primary amine, secondary amine, and carboxyhydrazide.

8. The method according to claim 1 wherein R2 is selected from the group consisting of: hydroxy, methoxy, ethoxy, propoxy, butoxy, and phenoxy.

9. An aqueous composition comprising: a) water; b) at least one oxidized polysaccharide containing aldehyde groups, said oxidized polysaccharide having a weight-average molecular weight of about 1,000 to about 1,000,000 Daltons and having an equivalent weight per aldehyde group of about 65 to about 1500 Daltons; and c) at least one oligomer of the formula:
R1-(PS)—R2
wherein:
(i) PS is a linear polymeric segment comprising ethylene oxide monomers or a combination of ethylene oxide and propylene oxide monomers, wherein said ethylene oxide monomers comprise at least about 50 weight percent of said polymeric segment;
(ii) R1 is at least one nucleophilic group capable of reacting with aldehyde groups to form at least one reversible covalent bond;
(iii) R2 is at least one functional group which is not capable of reacting with an aldehyde, a primary amine, a secondary amine, or R1 to form a covalent bond, such that said oligomer does not induce gelation when mixed in the aqueous solution with (b);
(iv) said oligomer has a weight-average molecular weight of about 200 to about 4,000 Daltons; and
(v) said oligomer is water soluble.

10. The aqueous composition according to claim 9 wherein the oxidized polysaccharide is selected from the group consisting of oxidized derivatives of: dextran, carboxymethyldextran, starch, agar, cellulose, hydroxyethylcellulose, carboxymethylcellulose, pullulan, inulin, levan, and hyaluronic acid.

11. The aqueous composition according to claim 10 wherein the oxidized polysaccharide is oxidized dextran.

12. The aqueous composition according to claim 9 wherein the oligomer is methoxy polyethylene glycol amine wherein PS is a linear polymeric segment derived from polyethylene oxide, R1 is a primary amine, and R2 is methoxy.

13. The aqueous composition according to claim 9 wherein the oxidized polysaccharide is present in said aqueous composition at a concentration from about 5% to about 40% by weight relative to the total weight of the aqueous composition.

14. The aqueous composition according to claim 9 wherein the oligomer is present in said aqueous composition at a concentration from about 0.5% to about 30% by weight relative to the total weight of the aqueous composition.

15. The aqueous composition according to claim 9 wherein R1 is selected from the group consisting of: primary amine, secondary amine, and carboxyhydrazide.

16. The aqueous composition according to claim 9 wherein R2 is selected from the group consisting of: hydroxy, methoxy, ethoxy, propoxy, butoxy, and phenoxy.
